(19) United States
(12) Patent Application Publication
(10) Pub. No.: US 2023/0322644 A1
(43) Pub. Date: Oct. 12, 2023

(54) ADDUCT COMPRISING AT LEAST A TRANSITION METAL AND AN ADDUCT OF A CARBON ALLOTROP AND A PYRROLIC COMPOUND

(71) Applicants: POLITECNICO DI MILANO, Milano (IT); CEA-COMMISARIAT A L'ENERGIE ATOMQUE ET AUX ENERGIES ALTERNATIVES, Gif-sur-Yvette Cedex (FR)

(72) Inventors: Maurizio Stefano Galimberti, Milano (IT); Vincenzina Barbera, Biancavilla (IT); Grégory Pieters, Gif-sur-Yvette Cedex (FR); Alberto Palazzolo, Gif-sur-Yvette Cedex (FR)

(21) Appl. No.: 18/020,976

(22) PCT Filed: Aug. 11, 2021
(86) PCT No.: PCT/EP2021/072396

§ 371 Date: Feb. 13, 2023

(30) Foreign Application Priority Data
Aug. 13, 2020 (IT) 102020000020104

(51) Int. Cl. C07B 59/00; C07D 215/06; B01J 23/46

(52) U.S. Cl. CPC C07B 59/002; C07D 215/06; B01J 23/462; C07B 2200/05

(57) ABSTRACT

The present invention relates to an adduct comprising at least one transition metal and an adduct between a sp² carbon allotrope and a pyrrole compound. In particular, the invention relates to an adduct comprising at least one transition metal and hydrophylic adducts between a sp² carbon allotrope and a pyrrole compound. Such adduct is preferentially used as catalytic system in a chemical reaction such as C—H activation, in particular the Hydrogen Isotope Exchange with isotopes such as deuterium and tritium.

The present invention relates to an adduct comprising at least one transition metal and an adduct between an sp² carbon allotrope and a pyrrole compound.

In particular, the invention relates to an adduct comprising at least one transition metal and hydrophylic adducts between an sp² carbon allotrope and a pyrrole compound.

The transition metal preferentially belongs to the class of late transition metals: iron, cobalt, nickel, ruthenium, rhodium, palladium, osmium, iridium, platinum. Other transition metals which can be used are: chromium, molybdenum, niobium, rhenium, tantalum, zirconium.

The preferred transition metals are: nickel, ruthenium, rhodium, palladium, iridium, platinum.

Such adduct is preferentially used as catalytic system in a chemical reaction such as C—H activation, in particular the Hydrogen Isotope Exchange with isotopes such as deuterium and tritium.

Catalysis is defined as the action of a catalyst which is a substance that increases the rate of a reaction without modifying the overall standard Gibbs energy change in the reaction.

It is widely acknowledged the importance of catalysts for chemical reactions. Some 90% of all the products available on the commercial scale involve catalyzed reactions, at least in a production step. Catalysts increase the speed of chemical reactions, by increasing both conversion and selectivity. Indeed, catalysts are able to selectively increase the rate of one of the many thermodynamically possible reactions. Catalysts allow thus to have a high atom efficiency (Pure Appl. Chem., Vol. 72, No. 7, pp. 1233-1246, 2000), which is the product of atom economy and reaction yield. A catalyst is a substance which is present at the beginning and at the end of the reaction and thus is not consumed during the reaction. The best catalysts maintain their efficiency over long time and many reaction cycles. As reported in “IUPAC. Compendium of Chemical Terminology, 2nd ed. (the “Gold Book”). Compiled by A. D. McNaught and A. Wilkinson. Blackwell Scientific Publications, Oxford (1997)”, catalysis can be classified as homogeneous catalysis, in which only one phase is involved, and heterogeneous catalysis, in which the reaction occurs at or near an interface between phases. Homogenous catalysts usually achieve high activity and selectivity, are applied to a large variety of chemical reactions, allow an efficient heat transfer and also the study of the reaction mechanisms. However, in the case of acid/base catalysts, they are often toxic (or corrosive) and are contaminated by the reaction products, whose separation can be troublesome, implying for example a distillation step. In such type of catalysis, recyclability is definitely a problem and production processes are mainly batchwise. Heterogeneous catalysts allow an easier separation from the reaction products and give rise to a lower amount of wastes. Catalysts are more recyclable and reusable, are definitely less toxic (or corrosive). Processes can be continuous and can be performed in many different types of reactors. However, applications are limited, with respect to those of the homogeneous catalysts, in particular because, in general, they have lower activity and lead to lower selectivity, as they have multiple catalytic sites, which are difficult to be identified, described and quantified.

The use of catalytic reactions has as main objective to avoid wastes, a major problem in the light of the goals of a sustainable development. In Pure Appl. Chem. (Vol. 72, No. 7, pp. 1233-1246, 2000) it is written “a primary cause of waste generation is the use of stoichiometric inorganic reagents”, “The solution is simple: replacement of stoichiometric methodologies with cleaner, catalytic alternatives.” Hence, it would be highly desirable to have a heterogenous catalyst with high catalytic activity and selectivity, so to allow achieving a high atom efficiency.

As it is written in JOC: “C—H functionalization represents a paradigm shift from the standard logic of organic synthesis. Instead of focusing on orchestration of selective reactions at functional groups, the new logic relies on the controlled functionalization of specific C—H bonds, even in the presence of supposedly more reactive functional groups. This family of reactions occurs thanks to the so called C—H activation. As it was written in (Nature 446, 391-393; 2007) “The stability of the chemical bonds in saturated hydrocarbons makes them generally unreactive.” “The invention of processes in which carbon-hydrogen (C—H) bonds in hydrocarbons can be activated is allowing chemists to exploit organic compounds in previously unimaginable ways”. “These reactions could revolutionize the chemical industry.” “C—H activation also allows chemical groups to be placed directly in a molecule where none existed before, a process that previously often needed several steps. This is especially useful for shortening multi-step syntheses, which are commonly used in drug discovery.” Examples of chemical bonds which can be introduced in place of C—H are: groups are: C—C, C—N, C—O, C—B. In the 90's “a number of metal salts and complexes were found to initiate C—H activation by oxidative addition. But the drawback was that most of these transformations required equal amounts, in moles, of the hydrocarbon and the metal, and both partners were consumed during the reaction. This is not acceptable for large-scale chemistry, as the metals involved are generally more expensive than the products.”

The solution of this problem was to use catalytic reactions. Again in (Nature 446, 391-393; 2007) it was written: “There has been an explosion of interest in the use of catalytic reactions for bringing about oxidative addition for C—H activation.”

The replacement of H in the C—H bond can be realized with another isotope of hydrogen. It is known that different isotopes exist for the atom which has only one proton in the nucleus and only one electron, that means for the hydrogen atom. They are: protium, deuterium and tritium, which have zero, one and two neutrons in the nucleus, respectively. Protium, deuterium and tritium are thus the three isotopes of the hydrogen atom. Usually, these three isotopes are simply called: hydrogen, deuterium and tritium. Deuterium and tritium are known as heavy hydrogen. Atoms of heavy hydrogen, hence atoms of deuterium or tritium, can be in place of hydrogen (protium) atoms in organic compounds. In particular, carbon atoms can be bound to deuterium or to tritium instead of hydrogen.

The organic compounds which contain carbon-(heavy hydrogen) bonds are greatly important. As it was written in (Angew. Chem. Int. Ed. 2007, 46, 7744-7765) “The increasing demand for stable isotopically labeled compounds has led to an increased interest in H/D-exchange reactions at carbon centers.” As reported in (Chemical Communications, 46(27), 4977-4979, 2010), multi-deuterium labeled compounds are utilized as tracers or surrogate compounds for the analyses, for example, of drug metabolism and environmental pollutants, deuterated medicines are new drug candidates, multi-deuterated alkanes are internal markers to prevent the distribution of illegal light diesel, regioselectively deuterium-labeled compounds are used in the investigation of chemical reaction mechanisms and kinetics and the structural elucidation of biological macromolecules such as sugars and peptides. Moreover, fully-deuterated polymers are useful as materials for optical fibers for high-speed telecommunication, as they are virtually free of any optical absorption based on the C—H stretching vibration. Concerning the analytical investigations, a carbon-deuterium bond can be distinguished from a carbon hydrogen (protium) bond. For example, in a ¹³C-NMR spectrum, an upfield shift is obtained by replacing a C—H bond with a C-D bond. In the pharmaceutical field, deuterated or tritiated compounds are usually used as contrast enhancers for drug discovery, as they give very important information about the biological behavior of the drugs and of their metabolites. Chemical compounds containing tritium atoms are used as markers for tests in vivo. The different behavior due to the isotopic effect is characteristic also of other applications, such as pesticides and electroluminescent organic materials. As a recent development of heavy drugs, a deuterium atom is introduced in the metabolic site of a drug to increase is lifetime, allowing to reduce the dose and therefore the side effects. Results are reported in https://doi.org/10.4155/fmc-2019-0183 and https://doi.org/10.1002/anie.201704146.

Relevant research is thus performed, in both academic and industrial fields, on organic compounds containing heavy hydrogen atoms, starting from their preparation. Focus of the research was and is on compounds containing carbon-deuterium bonds.

For the preparation of chemical compounds containing carbon-deuterium bonds, the synthesis can start from precursors which already contain deuterium atoms. However, as it was written in (Angew. Chem. Int. Ed. 2007, 46, 7744-7765) “Access to these deuterated compounds takes place significantly more efficiently and more cost effectively by exchange of hydrogen by deuterium in the target molecule than by classical synthesis.” Hence, the preferred synthetic pathway leads to the preparation of target chemical compounds which contain only carbon-hydrogen bond, performing then the exchange reaction between hydrogen and deuterium. It is thus preferred the so-called isotopic exchange: a covalently bonded hydrogen atom is replaced by a deuterium atom. Moreover, the preferred approach is to perform such isotopic exchange in the presence of a catalyst. The following catalysis are employed: acid catalysis, base catalysis, homogenous metal catalysis, heterogenous metal catalysis.

The pH dependent catalysis is the oldest one. Typically, D₂O or deuterated alcohols are used as deuterium source. As reported in (Angew. Chem. Int. Ed. 2007, 46, 7744-7765), the reverse exchange of deuterium for hydrogen can take place and “further chemical steps are often necessary to achieve deactivation.” In general, more reaction steps and reagents are required.

The advantages of H/D-exchange reactions with soluble transition metal complexes are nicely summarized in (Angew. Chem. Int. Ed. 2007, 46, 7744-7765) and in (Angew. Chem. Int. Ed. 2018, 57, 3022-3047): mild reaction conditions, high tolerance towards a number of functional groups, very efficient deuterium incorporation and often concomitant high regioselectivity. These catalysts are also suitable for the incorporation of tritium. Deuterium gas, deuterium oxide and deuterated solvents are used as deuterium source. Complexes are based on late transition metals such as Iridium, Rhodium, Rutenium, Cobalt and Platinum.

The drawbacks of a homogenous catalysis have been mentioned above. Moreover, the homogeneous catalytic H/D exchange with platinum has been restricted to tetrachloridoplatinate(II) salts. Specific acidic conditions are required, unless the reaction is microwave assisted. Hence a technique difficult to scale up is required. Moreover, the complexes to be used as catalyst have to be synthesized, not through trivial chemical pathways. In (JACS 1999, 121(18), 4385-4396), complexes are [C₅Me₅Rh(CH₂CHR)₂], in which the olefin bears a bulky silyl R substituent: SiMe₃, SiMe₂OEt, Si(Oi Pr)₃, SiMe(OSiMe₃)₂, SiPh₂O. In (J. Am. Chem. Soc. 2006, 128, 3974-3979) complexes are N-heterocyclic iridium-carbenes.

With heterogenous catalysts, high activity for H/D exchange was found (see Angew. Chem. Int. Ed. 2007, 46, 7744-7765) with palladium, platinum, rhodium, nickel, and cobalt catalysts. Combinations of catalyst and deuterium source were: Pd/C-D₂, Pd/C—H₂(D₂)/D₂O(DCl), Pd/C-DCO₂K, PtO₂/D₂/D₂O, Rh/SiO₂/D₂, and Raney-Ni-D₂O. Information on H/D exchange reactions is available also in (Chemical Communications, 2010, 46(27), 4977-4979), where the multideuteration methods for aromatic compounds, aliphatic ketones and secondary alcohols are commented to occur when catalyzed by palladium or platinum on carbon (Pd/C, Pt/C) and fully deuterated alkanes are prepared with rhodium on carbon (Rh/C) as the catalyst. In addition to gaseous deuterium, D₂O and deuterated protic solvents were used. Catalytic hydrogen-deuterium exchange process was also used for labelling polymers such as polyethylenes, as reported in (Polymer, 102, 99-105, 2016) with rhenium catalyst supported on ultrawide-pore silica, working however under pretty hard conditions: deuterium partial pressure of 500 psi and a temperature of 170° C. for 17 h in isooctane. Up to 2007 high activity was not reported with Rutenium based heterogenous catalysts (see Angew. Chem. Int. Ed. 2007, 46, 7744-7765). In (Chemical Communications, 2010, 46(27), 4977-4979), it is reported the regio- and stereoselective H-D exchange reaction of sugars using the Ru/C as the catalyst with H₂/D₂O combination under mild reaction conditions.

Hence, from what reported in the literature, it appears that heterogenous catalysts based on late transition metals are effective in the H/D exchange reactions. In most cases, also on a commercial scale, heterogeneous catalysts with late transition metals are obtained by supporting the late transition metals on a carbon substrate. An example of the procedure for obtaining such heterogenous catalysts is described for example in U.S. Pat. No. 4,728,630, where a Rh/C catalyst is prepared by contacting a carbonaceous support material having a surface area of at least 600 m²/g and a pH value in its aqueous suspension of 9-11 with an aqueous rhodium salt solution having a pH of 1-4. In this case the catalyst is then used for purification of terephthalic acid under reducing conditions. CN 108993497 reported a nano-ruthenium carbon catalyst for the hydrogenation of aromatic ring compounds. The preparation method comprises many steps: activated carbon is soaked in an aqueous solution of ruthenium salt; a basic compound is added; stirring, putting still, filtering, drying and calcination are conducted to obtain a first ruthenium carbon precursor; then the first ruthenium carbon precursor is soaked in an auxiliary aqueous solution; then the basic compound is added; stirring, putting still, filtering and drying are conducted to obtain a second ruthenium carbon precursor; under a protective atmosphere, the second ruthenium carbon precursor is subjected to a staged calcination treatment to obtain a third ruthenium carbon precursor; and then a reduction treatment is conducted to obtain the catalyst.

Metal/C catalysts are used also for H/D exchange reactions. EP 0276675 reports a method for the production of deuterated organic compounds by replacement of the hydrogen with deuterium in the liquid phase or gas phase. The exchange reaction is performed with D₂O at a temperature of from 150 to 350° C., preferentially from 200° C. to 300° C. in the gas phase. The exchange reaction (on acrylic chemicals) is performed in the presence of a supported catalyst based on palladium, nickel, copper or copper-chromium oxide. The support can be SiO₂, carbon black, activated charcoal and silicates.

Many methods to support the transition metals on Carbon are reviewed in (Nanoscale Research Letters (2018) 13:410). Basically, they are: impregnation, reduction, electrodeposition. In (Scientific Reports 2020, 10:7149), the supportation of Pt and Ru on carbon black is described as follows: a solution containing 50 mL water/ethyl alcohol mixture, 2.5 mmol Vulcan carbon, and 0.25 mmol K₂PtCl₄ and 0.25 mmol RuCl₃·3H₂O was prepared. The resulting solution was refluxed at 90° C. for 2 h. The catalyst was filtered and washed. In many cases, chemical reactions involving also refluxing and filtration steps have to be performed. Hence, usually for supporting the transition metal on a carbon support, carbon black with high surface area is used but specific chemical modification of the carbon substrate for the attachment of transition metal particles are not adopted.

The anchoring of the transition metal particles on a carbon substrate would be highly beneficial to ensure the stability of the catalytic system and to enhance the catalytic performance, as commented fro example in (Duan, 2017). It is worth repeating and emphasizing that the advantages of heterogenous catalysis can be really exploited only if the selectivity of the process is high, so non side reactions occur. This means not only that the atom efficiency is high but also that no further work-up steps are necessary. In this respect, larger catalytic efficiency is definitely desirable. Moreover, the catalytic system should work without adopting harsh experimental conditions, ideally avoiding high reaction temperatures.

As also commented in (Duan, 2017), to enhance the catalytic efficiency, synergy is pursued between the morphology of the substrate, the size of the catalyst particles and the encoring of the particles on the substrate. In fact “metal NPs in supported catalysts are apt to aggregate because of their large surface-to volume ratio”.

In order to emphasize the surface area of the substrate, sp2 carbon allotropes with particular morphology and nanosized carbon allotropes such as graphene layers and carbon nanotubes (CNT) have been recently used. It is widely acknowledged that such nanosized carbon allotropes have exceptional mechanical and electrical properties and this was also a reason for their selection. The scientific publications reveal a way to support the transition metal atoms on the nanosized carbon allotropes. The adducts were not used for the H/D exchange reaction.

In (Chen, 2016), Pd nanoparticles metal NPs encapsulated into the shell of nitrogen doped hollow carbon spheres. The procedure involved the synthesis of the hollow spheres and the impregnation of a palladium salt.

In (Duan, 2017), ultrafine palladium nanoparticles were supported on nitrogen-doped carbon microtubes. The preparation of the N-doped microtubes was the following: an aluminosilicate fiber was impregnated with dopamine, polymerized to polydopamine, carbonization was performed by treatment in Ar at 900° C., etching was done with HF solution. Pd particles were then in situ grown by using K₂PdCl₄ as the precursor, with the NCT layer as both the reducing and capping agent.

In the case of CNT, the adduct with the transition metal was made with nonfunctionalized or functionalized CNT. In (Electrochem Commun 2006; 8:1445-52), CNT were used without a functionalization step: CNT directly grown on carbon cloths underwent a hydrophilic treatment at 50 mV s⁻¹ for 100 cycles with potential ranged from 0.25 to +1.25 VSCE in an 02 saturated 2 M H₂SO₄ aqueous solution and electrochemical deposition of platinum and platinum-ruthenium nanoparticles was performed. The need for the functionalization is explained in (J. Phys. Chem. C 2009, 113, 1466-1473) and is in line with what reported above: “For the preparation of Pt-CNT nanocomposites, the functionalization of CNTs is an important step. The attachment of Pt nanoparticles on nonfunctionalized CNTs is found to be insignificant. Not only is the surface functionalization important for the attachment of nanoparticles, but also the catalytic activity is related to the active sites on the carbon surfaces” So, CNT having diameters between 110 to 170 nm and lengths of 5-9 μm are functionalized by sonicating them in the mixture of concentrated HNO₃ and concentrated H₂SO₄ (1:3 ratio) for 1 h. In a typical experiment, 30 mg of hexachloroplatinic acid is mixed with 400 μL of oleyl amine and 200 μL of oleic acid in 50 mL of dry toluene. The mixture is refluxed at 120° C. for different times (typically 1 to 6 h) under vigorous stirring. Reduction of the Pt salt is then performed with sodium borohydride. This catalyst is used for its electrocatalytic activity for methanol oxidation.

In (Duan, 2017), it was written “introduction of uniform N dopants into carbonaceous supports should be an effective route to anchor uniformly dispersed metal NPs as well as decrease their size distribution, and thus lead to an enhancement in the catalytic performance”. In Electrochemistry Communications 9 (2007) 1145-1153, an adduct of polypyrrole (PPy) and multiwalled CNT was formed by in situ polymerization of pyrrole on carbon nanotubes in 0.1 M HCl containing (NH₄)S₂O₈ as oxidizing agent over a temperature range of 0-5° C. Pt nanoparticles were deposited on PPy-CNT composite films by chemical reduction of H2PtC16 using HCHO as reducing agent at pH=11. It was shown that Pt—Ru/PPy-CNT catalysts exhibited higher catalytic activity for methanol oxidation. In (Applied Catalysis B: Environmental 80 (2008) 286-295), N-containing CNT supported electrodes were used. CNT were prepared by carbonizing poly(paraphenylene) tubules formed inside an alumina template. A multistep process is adopted, which involves cationic benzene polymerization, washing of catalyst, and carbonization of the tubules. The process in the presence of p-vinyl-pyrrolidone led to N-doped CNT. It was reported that the N-containing CNT favours better dispersion of Pt particles and that the strong metal-support interaction plays a major role in enhancing the catalytic activity for methanol oxidation.

Transtion metal catalyst particles have been also supported on graphene layers.

In (Xu, 2014), palladium and ruthenium molecular complexes containing an N-heterocyclic carbine ligand with a pyrene tag were anchored on the surface of reduced graphene oxide (rGO) by π-stacking. The procedure starts from the synthesis of the imidazolium complex, then involves the preparation of the adducts with transition metal complexes and finally the immobilization of graphene oxide, which has to be prepared from graphite. Palladium catalyzed hydrogenation of alkenes and ruthenium-catalyzed alcohol oxidation were performed.

In (Nature Communications, 2017, 8(1), 1-12), RuCo nanoalloys were encapsulated in nitrogen-doped graphene layers, by moving from Ru-doped Prussian blue analogues. The as-prepared Co₃[Co(CN)₆]₂ nanoparticles (nanocubes) were dispersed in a distilled water under agitated stirring to get a homogeneous mixed solution, followed by the addition of RuCl₃ solution (Ru doped nanocube). After agitated stirring for 10 h in dark, the brown products were collected and rinsed several times by distilled water, and finally dried under oven at 60° C. The thermal decomposition of the MOF precursor was performed at 600° C. for 4 h under nitrogen atmosphere in the oven with a heating rate of 10° C.min-1. Reduction with NaBH₄ led to Ru nanoparticles. Aggregates of RuCo alloys encapsulated in graphene layers were obtained. Such compounds were used as active electrocatalysts for producing hydrogen in alkaline media.

In (Acs Nano, 11(7), 6930-6941, 2017), atomically dispersed Ru on nitrogen-doped graphene were synthesized by means of NH₃ atmosphere annealing of a graphene oxide (GO) precursor containing trace amounts of Ru. The preparation is not simple. It starts from GO, whose synthesis is indeed troublesome, as it involves harsh reaction conditions and hazardous reagents. Moreover the procedure implies many steps. To mention only the firsts: a solution of metal salt was prepared using Ru(NH₃)₆Cl₃ (for the Ru catalyst) or FeCl₃ (for the Fe catalyst), respectively. The metal salt solution was added into the GO solution, and the mixture was sonicated. This precursor solution was freeze-dried for at least 24 h to produce a precursor foam. The temperature was ramped up to 750° C., while feeding Ar and NH₃ at room pressure. The reaction was carried out for 1 h, then the furnace was cooled to room temperature under Ar protection and the metal/nitrogen-doped graphene was then obtained. This catalytic system was used for oxygen reduction reaction in acidic medium.

The examples reported in the prior art for the supportation/encapsulation of transition metal particles on hollow carbon spheres, CNT and graphene layers demonstrate the beneficial effect of the substrate functionalization, which is however achieved through complex and also troublesome multistep reactions. Moreover, these catalysts were not used for C—H activation reactions and, in particular, for H/D exchange reactions.

It would be highly desirable to have heterogenous catalysts able to perform H/D exchange reactions with high yield, that means with high conversion and particularly high selectivity, avoiding thus wastes and work up procedures.

It would be desirable that the H/D exchange reaction could be performed under mild conditions.

It would be indeed desirable that the catalyst could be used for more reaction cycles.

It would be indeed desirable that the products of the reaction could be easily isolated from the reaction medium.

It would be indeed desirable that the support for the catalyst was a sp² carbon allotrope, preferentially: furnace carbon black, carbon nanotubes, nanosized graphite, graphene and graphene related materials.

It would be really desirable that the catalyst could be prepared with a simple and sustainable procedure, ideally in few steps, avoiding harsh reaction conditions and ideally, wet chemistry reactions based on organic solvents.

In particular, it would be desirable, for the preparation of the catalyst, to avoid the use of toxic and hazardous solvents, such as for example halogenated and/or aromatic solvents.

It would be desirable that the solvent, when used, could be water.

It would be desirable that the catalyst could be easily isolated from the process for its preparation.

An object of the present invention is to obtain heterogenous catalysts able to perform H/D exchange reactions with high yield, that means with high conversion and particularly high selectivity, avoiding thus wastes and work up procedures.

A further object is to perform the H/D exchange reaction under mild conditions.

A further object of the present invention is that the catalyst could be used for more reaction cycles.

An object of the present invention is also that the products of the reaction could be easily isolated from the reaction medium.

An object of the present invention is to use, as the support for the catalyst, a sp² carbon allotrope, preferentially: furnace carbon black, carbon nanotubes, nanosized graphite, graphene and graphene related materials.

A further object of the present invention is to prepare the catalyst with a simple and sustainable procedure, ideally in few steps, avoiding harsh reaction conditions and ideally avoiding as well wet chemistry reactions based on organic solvents.

A particular object of the present invention is to prepare the catalyst avoiding the use of toxic and hazardous solvents, such as for example halogenated and/or aromatic solvents.

In the light of the previous object, ideally the solvent should be ecofriendly, such as an alcohol and ideally water.

An objective of the present invention is the easy isolation of the catalyst from the process for its preparation.

These and other objects of the present invention are achieved by means of an adduct according to claim 1.

In particular by means of an adduct of a transition metal selected from the group consisting of: iron, cobalt, nickel, ruthenium, rhodium, palladium, osmium, iridium, platinum. chromium, molybdenum, niobium, rhenium, tantalum, zirconium, or mixture thereof; with an adduct of:
a sp² carbon allotrope and/or its derivative and compound of formula (I)

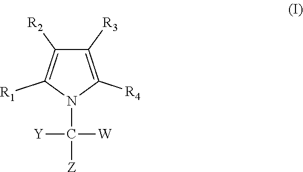

wherein R₁, R₂, R₃, R₄ are independently selected from the group consisting of: hydrogen, alkyl C₁-C₃, alkenyl or alkynyl C₂-C₆ linear or branched, aryl, alkyl-aryl C₁-C₆ linear or branched, alkenyl-aryl C₂-C₆ linear or branched, alkynyl-aryl C₂-C₆ linear or branched, heteroaryl, and Y, Z e W are independently selected from the group consisting of hydrogen, alkyl C₁-C₆, alkenyl or alkynyl C₂-C₆ linear or branched, or selected from the from the group consisting of:

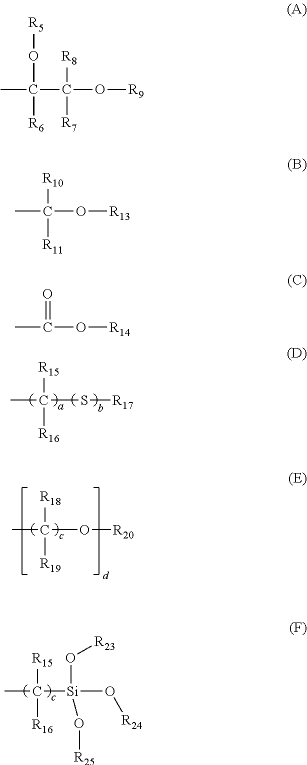

wherein R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, are independently selected from the group consisting of hydrogen, alkyl C₁-C₆, alkenyl or alkynyl C₂-C₆ linear or branched, aryl, alkyl-aryl C₁-C₆ linear or branched, alkenyl-aryl C₂-C₆ linear or branched, alkenyl-aryl C₂-C₆ linear or branched, heteroaryl and carboxyl, and wherein b is an integer from 1 to 4 and a, c, d and e are, independently, integers from 1 to 12.

With the adduct according to the present invention a large selectivity in the H/D exchange reaction is obtained.

Preferably said transition metal is selected from the group consisting of: nickel, ruthenium, rhodium, palladium, iridium, platinum or mixture thereof.

Preferably R₁, R₂, R₃, and R₄ are independently selected from the group consisting of: H, CH₃, CH₂CH₃, CH₂CH₂CH₃, phenyl.

Preferably said carbon allotrope or its derivative is selected from the group consisting of: carbon black, fullerene, Buchminstefullerenes, carbon nanohorns, carbon nanotubes, single-walled or multi-walled, carbon nanobuds, graphene, bilayer graphene, few-layer graphene, graphenylene, ciclocarbons, graphites with a number of stacked graphene layers from 2 to 10000.

Preferably said carbon allotrope derivative contains functional groups selected from the group consisting of:
functional groups containing oxygen, preferably hydroxyls, epoxies;
functional groups containing carbonyls, preferably aldehydes, ketones, carboxylic acids;
functional groups containing nitrogen atoms, preferably amines, amides, nitriles, diazonium salts, imines;
functional groups containing sulfur atoms, preferably sulfides, disulfides, sulfinates, sulfoxides, mercaptans, sulfones, sulfinic, sulfoxylic, and sulfonic groups.

In this way a vast range of carbon allotropes is available.

Preferably said derivative of said carbon allotrope is graphite oxide.

Preferably said derivative of said carbon allotrope is graphene oxide.

A further object of the present invention is to provide process for the preparation of an adduct according to claim 1, comprising the steps of:
i. providing a solution and/or suspension of a compound of formula (I) in a protic or aprotic polar solvent;
ii. providing a mixture of the carbon allotrope in a protic or aprotic polar solvent used for the preparation of the solution and/or suspension referred to in step i.;
iii. mixing said solution and/or suspension (i) and said mixture (ii);
iv. stirring;
v. if necessary, removing said solvent from said mixture obtained in step iii;
vi. providing energy.
vii. if necessary, dispersing the obtained mixture in the protic or aprotic polar solvent;
viii. adding a salt of the transition metal soluble in the selected protic or aprotic polar solvent;
ix. stirring;
x. if necessary, removing said solvent from the obtained mixture.

The process optionally comprising the additional steps of:
xi. if necessary, dispersing the mixture obtained after step vi in the protic or aprotic polar solvent;
xii. adding a reducing agent;
xiii. stirring;
xiv. removing said solvent from the obtained mixture.

Preferably said reducing agent is selected from the group consisting of: alcohols, aldehydes, carboxylic acids,

Preferably said reducing agent is present in an equimolar amount respect to the transition metal salts.

Preferable reducing agents are selected from the group consisting of diols, triols and reducing sugars such as glucose, dextrose, fructose; hydrides such as NaBH₄, LiAlH₄; organic acids such as ascorbic acid, citric acid.

According to the present invention, the term reducing agent is referred to an agent that allows the complete transfer of one or more electrons to a molecular entity (also called ‘electronation’), and, more generally, the reverse of the processes described under oxidation (2) and (3). (PAC, 1994, 66, 1077. (Glossary of terms used in physical organic chemistry (IUPAC Recommendations 1994)) on page 1160 [Terms] [Paper] Cite as: IUPAC. Compendium of Chemical Terminology, 2nd ed. (the “Gold Book”). Compiled by A. D. McNaught and A. Wilkinson. Blackwell Scientific Publications, Oxford (1997). Online version (2019-) created by S. J. Chalk. ISBN 0-9678550-9-8. https://doi.org/10.1351/goldbook.) Oxidizing and reducing agents are key terms used in describing the reactants in redox reactions that transfer electrons between reactants to form products. This page discusses what defines an oxidizing or reducing agent, how to determine an oxidizing and reducing agent in a chemical reaction, and the importance of this concept in real world applications.

According to the present invention, an oxidizing agent, or oxidant, gains electrons and is reduced in a chemical reaction. Also known as the electron acceptor, the oxidizing agent is normally in one of its higher possible oxidation states because it will gain electrons and be reduced. Examples of oxidizing agents include halogens, potassium nitrate, and nitric acid.

According to the present invention, a reducing agent, or reductant, loses electrons and is oxidized in a chemical reaction. A reducing agent is typically in one of its lower possible oxidation states, and is known as the electron donor. A reducing agent is oxidized, because it loses electrons in the redox reaction. Examples of reducing agents include the earth metals, formic acid, and sulfite compounds.”

The adduct according to the present invention will be better illustrated through the examples set down below, which illustrate the operating steps of the process for the preparation of this adduct.

Characteristics and advantages of the invention will be more apparent from the description of preferred embodiments, shown by way of non-limiting example in the accompanying drawings, wherein:

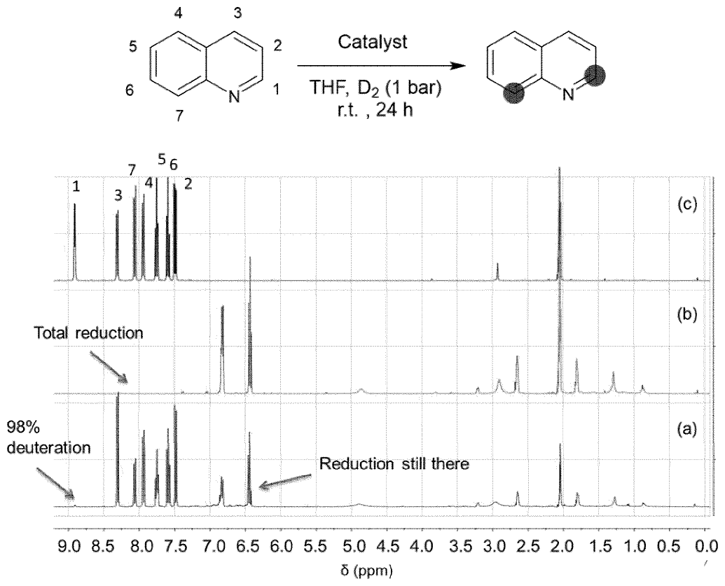

FIG. 1 shows the ¹H NMR spectra (400 MhZ, CDCl₃) for the deuteration of quinoline by using: (a) catalyst and reaction conditions as reported in example 19 (invention); (b) commercial ruthenium on carbon catalyst (RU/C); ¹H NMR spectrum of pure chinolin is also reported (c).

### EXAMPLES

### Materials

Reagents and solvents are commercially available and were used without any further purification: Serinol and isoserinol were kindly provided by Bracco. 2,5-hexandione, oxalyc acid, Ruthenium(III) chloride trihydrate, 1,2-propandiol, Ruthenium on carbon (Ru/C), quinoline and THF were purchased from Sigma-Aldrich.

Carbon Black N326 (CBN326) and N234 (CBN234) were from Cabot. Multiwall Carbon Nanotubes were NANOCYL® NC7000™ series, with carbon purity of 90%, average length of about 1.5 μm, BET surface area of 275 m²/g, 316 ml of absorbed DBP/100 grams of CNT. High surface area graphite (HSAG) was Nano24 from Asbury Graphite Mills Inc., with carbon content reported in the technical data sheet of at least 99 wt %. Chemical composition determined from elemental analysis was, as wt %: carbon 99.5, hydrogen 0.4, nitrogen 0.1, oxygen <0.05. BET surface area was 330 m²/g and DBP absorption was 162 mL/100 g.

Graphene Nanoplatelet (GnP) were from Sigma Aldrich.

In the following, are the examples on the preparation of adducts of sp² carbon allotropes (CA).

Examples 1-6 describe the preparation of adducts between sp² carbon allotropes and pyrrole compounds (PyC). They are named CA-PyC

Examples 7-12 describe the preparation of adducts between the adducts of sp² carbon allotropes with pyrrole compounds (CA-PyC) and Rutenium (Ru). These adducts are named CA-PyC/Ru. In these examples, methanol was used as solvent.

Examples 13-18 describe as well the preparation of adducts between the adducts of sp² carbon allotropes with pyrrole compounds (CA-PyC) and Rutenium (Ru). These adducts are named CA-PyC/Ru. In these examples 1,2-propandiol was used as solvent.

Examples 19-20 demonstrate the selectivity as catalyst of the adduct CA-PyC/Metal of the present invention. In particular, Example 19 demonstrates the selectivity as catalyst of the adduct CA-PyC/Ru as catalysts for the deuteration of quinolone. In Example 19, the CA-PyC/Ru adduct prepared in Example 7 was used. Example 20 demonstrates the reusability of the catalyst.

Example 21 is a comparative example. a commercial ruthenium on carbon catalyst (RU/C) was used.

### Examples 1-6: Preparation of Adducts Between Pyrrole Compounds (PyC) and Sp² Hybridized Carbon Allotropes (CA): CA-PyC

### Example 1: Adduct Between Multi-Walled Carbon Nanotubes (CNT) and 2-(2,5-dimethyl-1H-pyrrol-1-yl)propan-1,3-diol (SP)-CNT-SP

In a 50 mL flask, equipped with magnetic stirrer, CNT (200 mg, 2.8 mmol) and acetone (15 mL) were sequentially added. The thus obtained suspension was sonicated for 15 minutes using a 2 L ultrasound water bath. Afterwards, a solution of 2-(2,5-dimethyl-1H-pyrrol-1-yl)propan-1,3-diol (10% mol/mol, 0.28 mmol) in acetone (25 mL) is added to the suspension.

The mixture was then sonicated for 15 minutes. Afterwards, the acetone was removed under reduced pressure using a rotavapor. The black powder thus obtained was placed in a 100 mL flask and heated to 180° C. for 2 h. The adduct was then transferred in a Buchner filter and repeatedly washed with acetone (3×100 mL).

### Example 2: Adduct Between Graphene Nanoplatelets (GnP) and 2-(2,5-dimethyl-1H-pyrrol-1-yl)propan-1,3-diol (SP)-GnP-SP

GnP-SP was prepared with the procedure described in example 1, using graphene nanoplatelets instead of CNT.

### Example 3: Adduct Between High Surface Area Graphite (HSAG) and 2-(2,5-dimethyl-1H-pyrrol-1-yl)propan-1,3-diol (SP)-HSAG-SP

HSAG-SP was prepared with the procedure described in example 1, using high surface area graphite instead of CNT.

### Example 4: Adduct Between Carbon Black (CBN326) and 2-(2,5-dimethyl-1H-pyrrol-1-yl)propan-1,3-diol (SP)-CBN326-SP

CBN326-SP was prepared with the procedure described in example 1, using carbon black CBN326 instead of CNT.

### Example 5: Adduct Between Carbon Black (CBN234) and 2-(2,5-dimethyl-1H-pyrrol-1-yl)propan-1,3-diol (SP)-CBN234-SP

CBN234-SP was prepared with the procedure described in example 1, using carbon black CBN234 instead of CNT.

### Example 6: Adduct Between High Surface Area Graphite (HSAG) and 3-(2,5-dimethyl-1H-pyrrol-1-yl)propan-1,2-diol (iSP)-HSAG-iSP

HSAG-iSP was prepared with the procedure described in example 1, using 3-(2,5-dimethyl-1H-pyrrol-1-yl)propan-1,2-diol instead of SP.

### Examples 7-12: Preparation of Adducts Between Pyrrole Functionalized Carbon Allotropes (CA-PyC) and Rutenium (Ru) by Using Methanol as Solvent: CA-PyC/Ru

### Example 7: Preparation of the Adduct Between CNT-SP and Rutenium (CNT-SP/Ru)

In a 100 mL flask, equipped with magnetic stirrer, CNT-SP (200 mg) and methanol (50 mL) were sequentially added. The thus obtained suspension was sonicated for 10 minutes using a 2 L ultrasound water bath. Afterwards, Ruthenium(III) chloride trihydrate (10% mol/mol) and oxalic acid were added in sequence to the suspension. The mixture was then sonicated for 10 minutes. The so obtained mixture was heated for 3 hours at 70° C. Afterwards, the mixture was centrifugated at 4000 rpm for 30 minutes (3×10 mL methanol). The supernatant was removed and the black powder was dried.

### Example 8: Preparation of the Adduct Between GnP-SP and Rutenium (GnP-SP/Ru)

GnP-SP/Ru was prepared with the procedure described in example 7, using GNP-SP instead of CNT-SP.

### Example 9: Preparation of the Adduct Between HSAG-SP and Rutenium (HSAG-SP/Ru)

HSAG-SP/Ru was prepared with the procedure described in example 7, using HSAG-SP instead of CNT-SP.

### Example 10: Preparation of the Adduct Between CBN326-SP and Rutenium (CBN326-SP/Ru)

CBN326-SP/Ru was prepared with the procedure described in example 7, using CBN326-SP instead of CNT-SP.

### Example 11: Preparation of the Adduct Between CBN234-SP and Rutenium (CBN234-SP/Ru)

CBN234-SP/Ru was prepared with the procedure described in example 7, using carbon black CBN234-SP instead of CNT-SP.

### Example 12: Preparation of the Adduct Between HSAG-iSP and Rutenium (HSAG-iSP/Ru)

HSAG-iSP/Ru was prepared with the procedure described in example 7, using HSAG-iSP instead of CNT-SP.

### Examples 13-18: Preparation of Adducts Between Pyrrole Functionalized Carbon Allotropes (CA-PyC) and Rutenium (Ru) by Using 1,2-Propandiol as Solvent: CA-PyC/Ru

### Example 13: Preparation of the Adduct Between CNT-SP and Rutenium (CNT-SP/Ru)

CNT-SP/Ru was prepared with the procedure described in example 7, using 1,2-propandiol instead of methanol as solvent.

### Example 14: Preparation of the Adduct Between GnP-SP and Rutenium (GnP-SP/Ru)

GnP-SP/Ru was prepared with the procedure described in example 7, using 1,2-propandiol instead of methanol as solvent and GnP-SP instead of CNT-SP.

### Example 15: Preparation of the Adduct Between HSAG-SP and Rutenium (HSAG-SP/Ru)

HSAG-SP/Ru was prepared with the procedure described in example 7, using 1,2-propandiol instead of methanol as solvent and HSAG-SP instead of CNT-SP.

### Example 16: Preparation of the Adduct Between CBN326-SP and Rutenium (CBN326-SP/Ru)

CBN326-SP/Ru was prepared with the procedure described in example 7, using 1,2-propandiol instead of methanol as solvent and CBN326-SP instead of CNT-SP.

### Example 17: Preparation of the Adduct Between CBN234-SP and Rutenium (CBN234-SP/Ru)

CBN234-SP/Ru was prepared with the procedure described in example 7, using 1,2-propandiol instead of methanol as solvent and CBN234-SP instead of CNT-SP.

### Example 18: Preparation of the Adduct Between HSAG-iSP and Rutenium (HSAG-iSP/Ru)

HSAG-iSP/Ru was prepared with the procedure described in example 7, using 1,2-propandiol instead of methanol as solvent and HSAG-iSP instead of CNT-SP.

### Examples 19-20: Selective Deuteration of Quinoline by Using CA-PyC/Ru as Catalyst

### Example 19: Selective Deuteration of Quinoline by Using CNT-SP/Ru as Catalyst

A Fisher Porter bottle (V=80 mL) is charged with 20 mg of CNT-SP/Ru, prepared as reported in example 7 (0.2 mol %) and placed under vacuum for 5 minutes. A solution of 0.2 mmol of quinoline in dry THF (0.1 M) is added onto the vessel under an Argon flow. The reaction medium is, thus, evacuated from the Argon gas and exposed to 3 cycles of D₂ (1 bar)—Vacuum of 10 minutes each. Then the Fisher Porter bottle is closed and the medium is left to stir for 24 hours at room temperature. Thereafter the Fisher Porter is opened and the gas phase evacuated, the mixture is dissolved in 10 additional mL of distilled THF and it is centrifugated 8000 rpm for 10 minutes in order to separate the catalyst from the organic medium. The selective deuteration of quinoline was evaluated by means of ¹H-NMR spectroscopy (FIG. 1a).

### Example 20: Selective Deuteration of Quinoline by Using the CNT-SP/Ru Catalyst Used in Example 19 (Reusability of Catalyst)

A Fisher Porter bottle (V=80 mL) is charged with 20 mg of CNT-SP/Ru, recovered from reaction mixture of example 19, (0.2 mol %) and placed under vacuum for 5 minutes. A solution of 0.2 mmol of quinoline in dry THF (0.1 M) is added onto the vessel under an Argon flow. The reaction medium is, thus, evacuated from the Argon gas and exposed to 3 cycles of D₂ (1 bar)—Vacuum of 10 minutes each. Then the Fisher Porter bottle is closed and the medium is left to stir for 24 hours at room temperature. Thereafter the Fisher Porter is opened and the gas phase evacuated, the mixture is dissolved in 10 additional mL of distilled THF and it is centrifugated 8000 rpm for 10 minutes in order to separate the catalyst from the organic medium. The selective deuteration of quinoline was evaluated by means of ¹H-NMR spectroscopy.

### Example 21 (Comparative): Deuteration of Quinoline by Using a Commercial Ruthenium on carbon catalyst (RU/C)

### Example 21

A Fisher Porter bottle (V=80 mL) is charged with 20 mg of Ru/C (Aldrich) (0.2 mol %) and placed under vacuum for 5 minutes. A solution of 0.2 mmol of quinoline in dry THF (0.1 M) is added onto the vessel under an Argon flow. The reaction medium is, thus, evacuated from the Argon gas and exposed to 3 cycles of D₂ (1 bar)—Vacuum of 10 minutes each. Then the Fisher Porter bottle is closed and the medium is left to stir for 24 hours at room temperature. Thereafter the Fisher Porter is opened and the gas phase evacuated, the mixture is dissolved in 10 additional mL of distilled THF and it is centrifugated 8000 rpm for 10 minutes in order to separate the catalyst from the organic medium. (FIG. 1b)

1. An adduct of a transition metal selected from the group consisting of: iron, cobalt, nickel, ruthenium, rhodium, palladium, osmium, iridium, platinum, chromium, molybdenum, niobium, rhenium, tantalum, zirconium, or mixture thereof; with an adduct of:
a sp² carbon allotrope and/or its derivative and compound of formula (I) 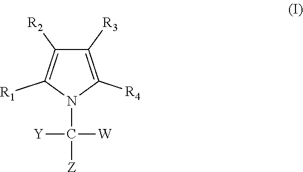
wherein R₁, R₂, R₃, R₄ are independently selected from the group consisting of: hydrogen, alkyl C₁-C₃, alkenyl or alkynyl C₂-C₆ linear or branched, aryl, alkyl-aryl C₁-C₆ linear or branched, alkenyl-aryl C₂-C₆ linear or branched, alkynyl-aryl C₂-C₆ linear or branched, and heteroaryl,
and
Y, Z, and W are independently selected from the group consisting of hydrogen, alkyl C₁-C₆, and alkenyl or alkynyl C₂-C₆ linear or branched,
or selected from the group consisting of: 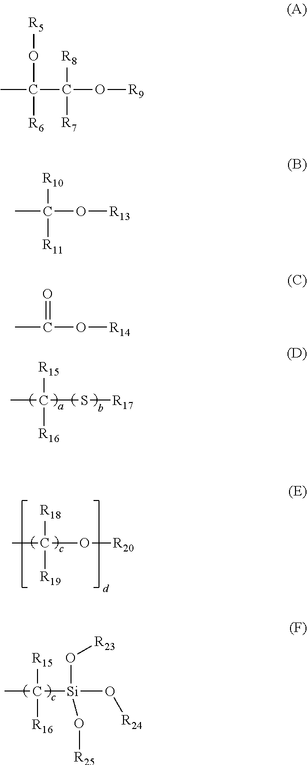
wherein R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, are independently selected from the group consisting of hydrogen, alkyl C₁-C₆, alkenyl or alkynyl C₂-C₆ linear or branched, aryl, alkyl-aryl C₁-C₆ linear or branched, alkenyl-aryl C₂-C₆ linear or branched, alkenyl-aryl C₂-C₆ linear or branched, heteroaryl, and carboxyl, and
wherein b is an integer from 1 to 4 and a, c, d and e are, independently, integers from 1 to 12.

2. The adduct according to claim 1, wherein the transition metal is selected from the group consisting of: nickel, ruthenium, rhodium, palladium, iridium, platinum and mixtures thereof.

3. The adduct according to claim 1, wherein the R₁, R₂, R₃, and R₄ are independently selected from the group consisting of: H, CH₃, CH₂CH₃, CH₂CH₂CH₃, and phenyl.

4. The adduct according to claim 1, wherein the carbon allotrope or its derivative is selected from the group consisting of: carbon black, fullerene, Buchminstefullerenes, carbon nanohorns, carbon nanotubes, single-walled or multi-walled, carbon nanobuds, graphene, bilayer graphene, few-layer graphene, graphenylene, ciclocarbons, and graphites with a number of stacked graphene layers from 2 to 10000.

5. The adduct according to claim 1, wherein the carbon allotrope derivative contains functional groups selected from the group consisting of:
functional groups containing oxygen, hydroxyls, epoxies;
functional groups containing carbonyls, aldehydes, ketones, carboxylic acids;
functional groups containing nitrogen atoms, amines, amides, nitriles, diazonium salts, imines;
functional groups containing sulfur atoms, sulfides, disulfides, sulfinates, sulfoxides, mercaptans, sulfones, sulfinic, sulfoxylic, and sulfonic groups.

6. The adduct according to claim 1, wherein the derivative of said carbon allotrope is graphite oxide.

7. The adduct according to claim 1, wherein the derivative of said carbon allotrope is graphene oxide.

8. A process for the preparation of an adduct according to claim 1, comprising the steps of:
i. providing a solution and/or suspension of a compound of formula (I) in a protic or aprotic polar solvent;
ii. providing a mixture of the carbon allotrope in a protic or aprotic polar solvent used for the preparation of the solution and/or suspension referred to in step i.;
iii. mixing said solution and/or suspension (i) and said mixture (ii);
iv. stirring;
v. if necessary, removing said solvent from the obtained mixture;
vi. providing energy;
vii. if necessary, dispersing the obtained mixture in the protic or aprotic polar solvent;
viii. adding a salt of the transition metal soluble in the selected protic or aprotic polar solvent;
ix. stirring;
x. if necessary, removing said solvent from the obtained mixture.

9. The process according to claim 8, further comprising:
xi. if necessary, dispersing the mixture obtained after step vi in the protic or aprotic polar solvent;
xii. adding a reducing agent;
xiii. stirring;
xiv. removing said solvent from the obtained mixture.

10. The process according to claim 9, wherein the reducing agent is selected from the group consisting of: alcohols, aldehydes, carboxylic acids.

11. The process according to claim 9, wherein the reducing agent is present in an equimolar amount with respect to the transition metal salts.
